(19) United States
(12) Patent Application Publication
(10) Pub. No.: US 2023/0175954 A1
(43) Pub. Date: Jun. 8, 2023

(54) ELECTRIC POWER ASSET HEALTH MONITORING

(71) Applicant: Qualitrol Company LLC, Fairport, NY (US)

(72) Inventors: Sri Krishna Bukya, Rochester, NY (US); Emilio Morales Cruz, Belton, TX (US); Anis Zribi, Rochester, TX (US); Andre Pelletreau, Fairport, NY (US); Colin Brown, Glasgow (GB); Paul Donegan, Dunoon (GB)

(21) Appl. No.: 18/061,929

(22) Filed: Dec. 5, 2022

Related U.S. Application Data
(60) Provisional application No. 63/286,501, filed on Dec. 6, 2021.

(51) Int. Cl. G01N 21/31

(52) U.S. Cl. CPC G01N 21/31; G01N 2201/08; G01N 2201/0635

(57) ABSTRACT

A method of directly measuring moisture content in an oil-filled transformer includes using an optical fiber that having a grating sensor, such as a Fiber Bragg grating, defined in the optical fiber. The various conductors (windings) in the transformer are insulated using an insulator such as paper and insulating oil is filled inside the transformer. Moisture in the transformer is absorbed by the paper that surrounds the windings. A moisture content at a specific location can be measured by placing the optical fiber with the grating sensor directly at the specific location to be measured. A physical parameter of the paper that absorbed moisture changes over time, causing a change in the grating sensor of the optical fiber which changes the spectral response of optical signals that are reflected by the grating sensor. The method provides an accurate method of measuring the moisture inside the transformer at the specific location.

### BACKGROUND

### Technical Field

The disclosure generally relates to directly measuring the amount of certain materials including moisture in an electrical transformer.

### Description of the Related Art

The presence of moisture in oil-filled transformers has one of the most significant effects on the short-term and long-term performance of an insulation system within the oil-filled transformers. Moisture is always present in the insulation system, to some degree, when it is shipped from the manufacturer due to residual moisture from manufacturing and will increase over the life of the transformer due to ingress from environment and as a byproduct of the aging process of the cellulose included in the insulation system. The presence of moisture in the insulation impacts transformer life, dielectric performance, and load limitations.

### BRIEF SUMMARY

Moisture in oil-filled electrical transformers is undesirable. The technical problem in the related art is how to determine how much moisture is present in the insulation system of the oil-filled transformers and more specifically, where in the complex insulation structure of the transformers that that moisture resides.

An electrical transformer often uses paper as an insulator (also referred to as ‘insulation paper’) for windings of the transformer. During operation of the transformer, moisture will move from the oil to the paper and vice versa depending on temperature and each material's moisture absorption characteristics. Practically, an operating transformer is never in an equilibrium condition regarding moisture distribution. This makes it very difficult to use equilibrium estimation methodology, measuring the moisture in one medium and estimating the moisture in the other medium. In most cases, the interest is in knowing the moisture content of the solid insulation at the hottest spot within a winding of the transformer.

Moisture content assessment in the insulation can be conducted directly, e.g., using Karl Fischer titration methodology on paper samples taken from an operating transformer which requires de-energization and exposure of the windings. Samples can be cut off from accessible areas such as leads and quickly wrapped up for shipping to a lab. However, taking paper samples from the interior of the transformer, especially from hot spot regions, is impractical. Other current methodologies in the related art for estimating the amount of moisture in the insulation system are indirect and do not provide an accurate value of moisture at the hottest spot within a winding. These methods in the related art are predicated on the assumption that, in a steady state condition, moisture will reach an equilibrium between the various materials contained within any closed vessel. The key assumption in these methodologies is that the materials are in equilibrium which often is not the case.

One or more embodiments of the present disclosure provide a system and method for directly measuring the moisture content in the insulation paper in an oil-filled transformer. The direct measuring system and method provides a technical benefit of accurately assessing the amount of moisture in the insulation paper of the oil-filled transformer. The direct measuring system and method does not rely on the key assumptions of the various methodologies in the related art and therefore is able to provide an accurate measurement of the amount of moisture at a specific location within the transformer. That is, the present disclosure provides a specific location calculation of the moisture content in the insulation paper rather than a general assessment calculation of the moisture content based on a measured moisture in the oil as currently employed in the related art.

In an embodiment, a method provides a fiber optic-based sensing element inside a transformer at a first location adjacent to an insulator wrapped around a winding of the transformer. The method transmits light through the fiber optic-based sensing element and senses optical signals based on light reflected by a grating sensor defined along a length of the fiber optic-based sensing element at the first location. The method also determines a moisture parameter of the insulator wrapped around the winding at the first location based on the sensed optical signals.

In an embodiment, a method provides a fiber optic-based sensing element inside an insulating oil of an oil-filled transformer. The fiber optic-based sensing element has a light- transmitting core and a hygroscopic material at least partially surrounding the light-transmitting core. The method transmits light through the fiber optic-based sensing element and senses optical signals that are reflected back from a grating sensor defined in the light-transmitting core at a location along a length of the fiber optic-based sensing element. The method also determines a moisture parameter of the insulating oil of the oil-filled transformer based on the sensed optical signals.

In an embodiment, a system, includes a first fiber optic-based sensing element at a first location inside an oil-filled transformer, a second fiber optic-based sensing element at a second location inside the oil-filled transformer, an interrogator operatively coupled to the first and second fiber optic-based sensing elements, and a processing circuitry coupled to the interrogator. The first fiber optic-based sensing element includes at least one set of Fiber Bragg gratings defined along a length of the first fiber optic-based sensing element at the first location. The second fiber optic-based sensing element includes at least one set of Fiber Bragg gratings defined along a length of the second fiber optic-based sensing element at the second location.

The interrogator is configured to transmit interrogating light to the first and second fiber optic-based sensing elements. The interrogator is further configured to receive optical signals based on light reflected back from the first and second fiber optic-based sensing elements.

The processing circuitry is configured to calculate a change in spectral response of the optical signals from a first point in time to a second point in time after the first point in time. The processing circuitry is further configured to determine a moisture parameter at the first location or at the second location based on the change in the spectral response of the optical signals.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless the context indicates otherwise. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Moreover, some elements known to those of skill in the art have not been illustrated in the drawings for ease of illustration. One or more embodiments are described hereinafter with reference to the accompanying drawings in which:

FIG. 1 is a diagram of a fiber optic-based sensing element placed inside a transformer according to various embodiments of the present disclosure.

FIG. 2 is an example configuration of a transformer and insulators wrapped around the windings according to various embodiments of the present disclosure.

FIG. 3 is an enlarged view of a fiber optic-based sensing element placed adjacent to a winding wrapped with an insulator as shown in FIG. 1.

FIG. 4 illustrates a fiber optic-based sensing element having a hygroscopic material around the fiber optic-based sensing element inside an oil-filled transformer.

FIG. 5 is a cross-sectional view of a fiber optic-based sensing element in a length direction including hygroscopic transducers coupled to the gratings inscribed in the core of the fiber optic-based sensing element according to various embodiments of the present disclosure.

FIG. 6 illustrates a fiber optic-based sensing element having a hydrogen sensitive material around the fiber optic-based sensing element inside an oil-filled transformer.

FIG. 7 is a cross-sectional view of a fiber optic-based sensing element having an insulator wrapped around and affixed to the gratings according to various embodiments of the present disclosure.

FIG. 8 illustrates a fiber optic-based sensing element using gratings for a pressure sensor according to various embodiments of the present disclosure.

FIG. 9 is a flow chart of a method according to various embodiments of the present disclosure.

FIG. 10 illustrates a system according to various embodiments of the present disclosure.

FIG. 11 illustrates an example monitoring system according to various embodiments of the present disclosure.

### DETAILED DESCRIPTION

The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to interfaces, physical component layout, etc., have not been shown or described in order to avoid unnecessarily obscuring descriptions of the embodiments. Additionally, the various embodiments may be methods, systems, or devices.

Throughout the specification, claims, and drawings, the following terms take the following meanings, unless the context indicates otherwise. The term “herein” refers to the specification, claims, and drawings associated with the current application. The phrases “in one embodiment,” “in another embodiment,” “in various embodiments,” “in some embodiments,” “in other embodiments,” and other variations thereof refer to one or more features, structures, functions, limitations, or characteristics of the present disclosure, and are not limited to the same or different embodiments unless the context indicates otherwise. As used herein, the term “or” is an inclusive “or” operator, and is equivalent to the phrases “A or B, or both” or “A or B or C, or any combination thereof,” and lists with additional elements are similarly treated. The term “based on” is not exclusive and allows for being based on additional features, functions, aspects, or limitations not described, unless the context indicates otherwise. In addition, throughout the specification, the meaning of “a,” “an,” and “the” include singular and plural references.

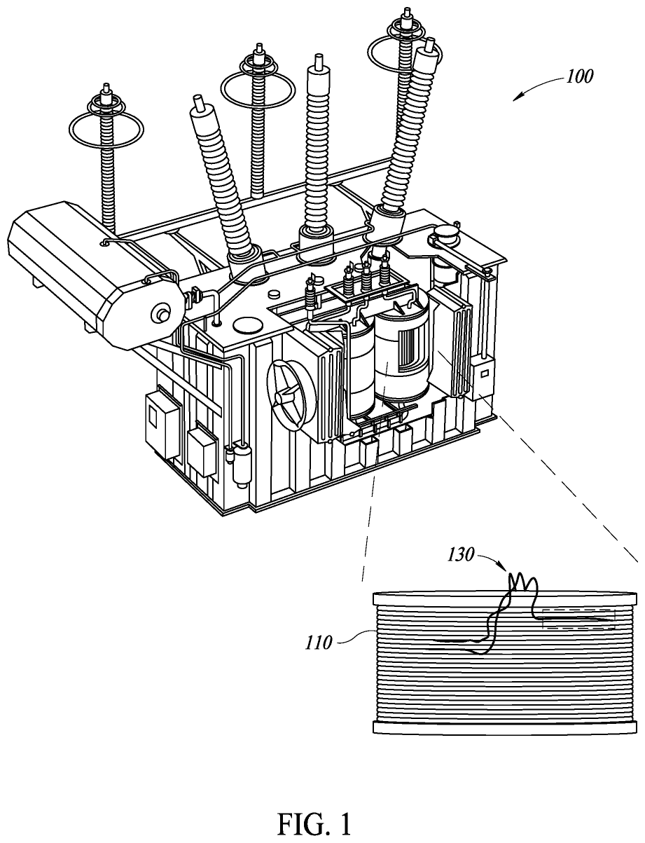

FIG. 1 is a diagram of a fiber optic-based sensing element placed inside an electrical transformer according to various embodiments of the present disclosure.

An electrical transformer is a device that transfers electric energy from one alternating-current circuit to one or more other circuits, typically either increasing (stepping up) or reducing (stepping down) the voltage. A transformer 100 as illustrated in FIG. 1 includes an oil-immersed transformer or an oil-filled transformer. An oil-filled transformer is a type of voltage transformation device that utilizes an oil cooling method to reduce the transformer temperature. Contrary to a dry type transformer, the body of an oil-filled transformer, including the core and the coils, is immersed in insulation oil. In operation, the heat of the coils and core is transferred to the insulation oil and the oil insulates and cools the coils and core.

In particular, the transformer 100 includes a magnetic core, windings 110 (e.g., coils), and bushings. The magnetic core provides a path for magnetic flow. The windings create a magnetic field and consist of a conductor coil wrapped around the core. The bushings connect the transformer windings to external electrical contacts, e.g., in a substation.

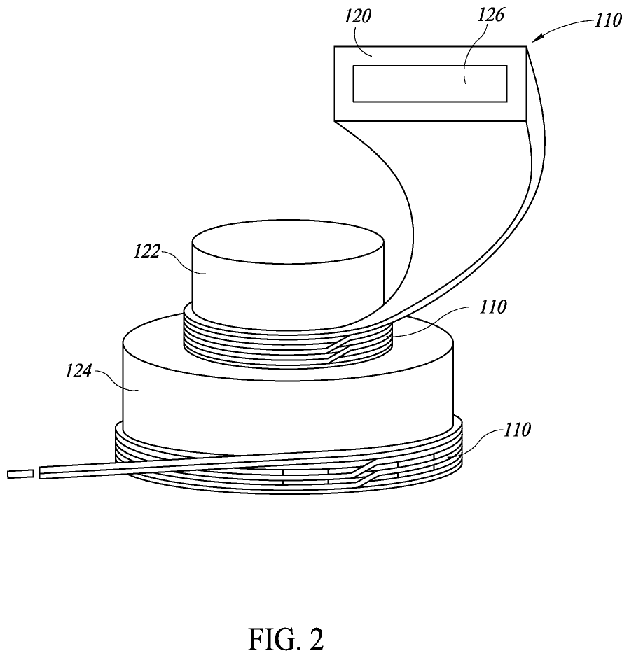

FIG. 2 is an example configuration of a transformer and insulators wrapped around the windings according to various embodiments of the present disclosure.

The transformer 100 includes a press board cylinder 122 and a cylinder 124. The windings 110 are wrapped around the press board cylinder 122. As shown, the windings 110 comprise a conductor 126 wrapped with an insulator 120. The insulator 120 provides mechanical and dielectric strength. The press board cylinder 122 separates the windings 110 from a core and the cylinder 124 separates high voltage windings from low voltage windings. In the figures, in some embodiments, when reference number 110 is used for windings, it includes the insulator 120 wrapped around the conductor 126.

Referring to FIG. 1, the windings 110 of the transformer 100 are wrapped using the insulator 120. A commonly used insulator material for the insulator 120 is a cellulose paper.

During the operation of the transformer, as the transformer heats up and is cooled, moisture in the oil-filled transformer will move from the oil to the paper and vice versa depending on temperature and each material's moisture absorption characteristics. Accordingly, directly measuring the moisture content in the insulator (here, the cellulose paper 120) provides an accurate measurement of the amount of moisture in the insulator at that particular location.

The various methods for measuring unwanted moisture in the insulation of an oil-filled transformer in the related art are, as mentioned above, mostly directed to an indirect measurement of the amount of moisture. For instance, this may include approximately measuring the amount of moisture in the oil itself as a proxy for estimating moisture in the paper insulator, rather than directly measuring the amount of moisture in the insulator. Further, due to their various assumptions (e.g., various materials contained within the transformer will reach an equilibrium) and the nature of their indirect measurement, the various methods in the related art provide a less reliable measurement of the amount of moisture in the insulator of the oil-filled transformer.

In order to provide a direct measurement of the moisture content of the insulator at a specific location, according to some embodiments, a fiber optic-based sensing element 130 is placed adjacent to the insulator of the windings 110 of the transformer 100. In various embodiments, the fiber optic-based sensing element 130 includes an optically-transmissive core (or a light-transmitting core) and a cladding surrounding the core. The fiber optic-based sensing element 130 also includes one or more gratings that function as a sensing element (i.e., grating sensor). The gratings are disposed along a length of the fiber optic-based sensing element 130. The gratings can be provided at various locations along the length of the fiber optic-based sensing element 130. Further, as shown in FIG. 1, more than one fiber optic-based sensing element 130 can be used to directly measure the moisture content (or moisture parameter indicative of moisture content) of the insulator of the windings at multiple locations. In addition, a fiber optic-based sensing element 130 can have different types of sensors along the length of the fiber optic-based sensing element 130. The gratings can be inscribed in the fiber optic-based sensing element 130 to implement these different types of sensors (e.g., pressure sensor, humidity sensor, temperature sensor, etc.). That is, in various embodiments, grating sensors can be implemented as pressure sensors, humidity sensors, temperature sensors, strain sensors, or the like. An example of these gratings include Fiber Bragg gratings (FBGs). However, the example is not limited to Fiber Bragg gratings.

The fiber optic-based sensing element 130, during operation, is placed sufficiently close to the insulator such that a change in the refractive index of the optical fiber due to the absorption or adsorption of moisture in the insulator can be sensed using the FBGs. For instance, when the insulator absorbs moisture, moisture causes changes in the physical or optical properties of the insulator which in turn induces changes in the optical fiber. In one embodiment, the fiber optic-based sensing element 130 is placed in immediate proximity to the insulator or is in direct contact with the insulator at a specific location to provide a direct and accurate measurement of the moisture parameter (one or more parameters indicative of moisture content) within the insulator at that location. The specific process of interrogating and receiving a spectral response (e.g., amplitude response, frequency response, or the like) from the FBGs to calculate the moisture content in the insulator will be detailed later herein.

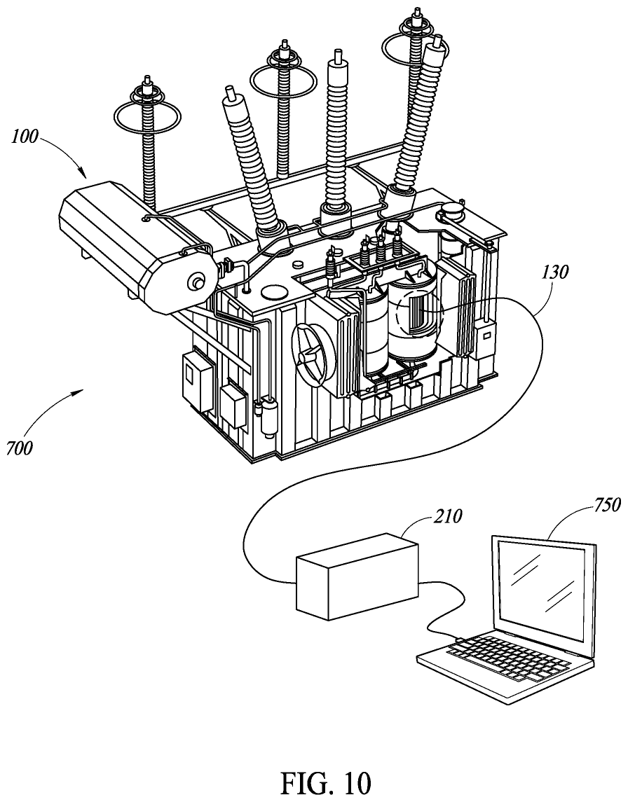

In various embodiments, the fiber optic-based sensing element 130 may be placed in the transformer during the manufacturing stage of the transformer. In these embodiments, the fiber optic-based sensing element 130 may be attached at specific locations of the transformer coil when the transformer is manufactured. This has some technical benefits as the manufacturer of a winding will likely know or at least anticipate the location of the hottest spot within the winding. Further, it will have a benefit of automatically providing spectral responses from the fiber optic-based sensing elements 130 to processing circuitry (see FIG. 10) via an interrogator (see FIG. 10) which can further lower the monitoring cost.

Finding the hottest spot within a winding (e.g., the highest temperature of the winding, or “winding hottest spot temperature”) is a prime concern for transformer operators. This variable is ideally known under all loading conditions, especially conditions involving rapid dynamic load changes. Accurate knowledge of the winding hottest spot temperature is a critical input for calculation of the insulation aging, assessment of the risk of bubble evolution, and short-term forecasting of the overload capability of the transformer. It is also critical for efficient control of the cooling banks to ensure that they can be set in motion quickly when needed.

Traditionally, the hottest spot temperature is provided by a winding temperature indicator (WTI) using a thermal image method. These devices typically rely on a measurement of the top-oil temperature and a simulation of the winding hottest spot temperature rise. An example instrument involves a bulb inserted in a thermowell and surrounded by insulating oil. To simulate the winding temperature, the thermowell is additionally fitted with a heater element, fed by a current proportional to the transformer loading.

This thermal image method has drawbacks, as it assumes that the temperature of oil at the top of the cooling duct is the same as the top oil temperature measured in an oil-filled thermowell near the top of the tank. This may be true under stable operating conditions but not necessarily under dynamic rapid load changes.

Accordingly, there are technical benefits of a direct measurement of winding temperature using a fiber optic-based sensing element 130 as it provides a more accurate approach for the determination of winding hot spot temperature during both stable loading conditions and dynamic loading conditions. That is, the use of a fiber optic-based sensing element 130 as described herein removes several uncertainties in the process of winding hot spot temperature determination and is not impacted by any loading conditions (both stable and dynamic loading conditions), which is an improvement compared to the methods used in the related art.

As described herein, an assessment of winding insulation temperature under any loading condition is a critical step for efficient management of an electrical transformer. The traditional model used for hot spot temperature determination has shown many limitations, especially under transient conditions. However, the advantages of the direct measurement method described herein, using a fiber optic-based sensing element 130 according to one or more embodiments of the present disclosure, is that it bypasses these difficulties and uncertainties. These fiber optic-based sensing elements can provide dependable information for each winding, under any loading condition. Further, a method according to one or more embodiments of the present disclosure can accurately measure moisture in the paper insulator using a fiber optic-based sensing element 130 as described herein (e.g., an FBG moisture sensor having, for example, a wholly polymeric fiber with a fiber grating in the cladding) mounted in the winding. This also allows determination of an accurate bubbling temperature calculation.

The FBGs in the fiber optic-based sensing element 130 are capable of measuring many parameters based on different applications. For instance, the FBGs can be used to sense pressure, temperature, humidity, strain, vibrations, liquid levels, hydrogen in transformers, and more, based on different spectral responses of the FBGs in reaction to different environmental conditions.

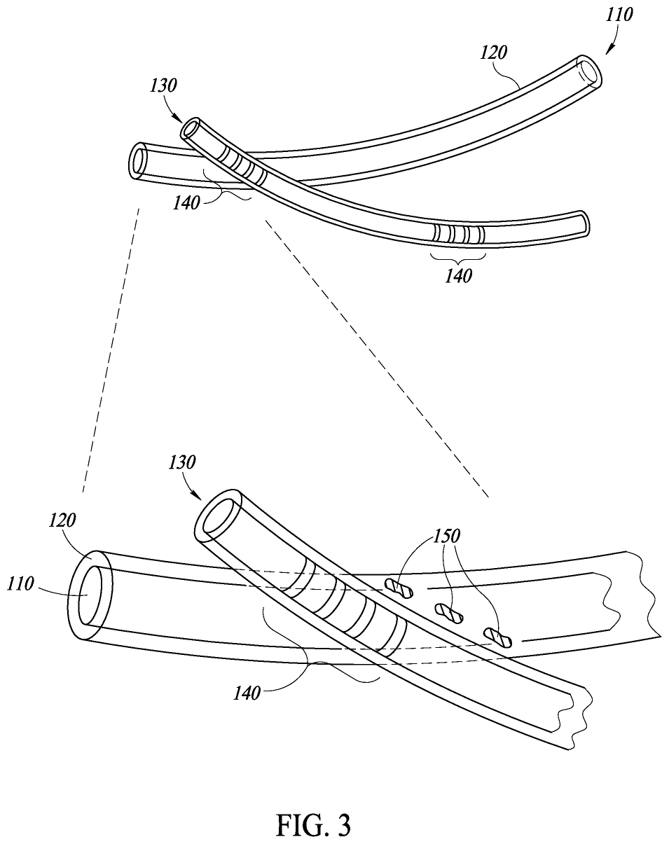

FIG. 3 is an enlarged view of a fiber optic-based sensing element 130 placed adjacent to a winding wrapped with an insulator as shown in FIG. 1.

In particular, FIG. 3 shows an enlarged view of how a fiber optic-based sensing element 130 is placed sufficiently adjacent to the insulator 120 that is wrapped around the winding 110, to sense the moisture content of the insulator 120 at that location.

During a normal operation of the transformer, moisture finds its way into the transformer, particularly into the insulation system of the transformer. Further, moisture is likely to increase over the life of the transformer due to ingress from environment and as a byproduct of the aging process of the cellulose in the insulation system. The presence of moisture in the insulation impacts transformer life, dielectric performance, and load limitations, and therefore obtaining an accurate measurement of moisture content within the insulator is beneficial.

FIG. 3 shows moisture 150 permeated and absorbed in the insulator 120 and further shows the FBGs 140 of the fiber optic-based sensing element 130 sufficiently close the insulator 120. Here, when an optical interrogator (see FIG. 10) transmits light into the fiber optic-based sensing element 130, a portion of the light leaks to the vicinity at the FBGs 140. Some part of the light is absorbed by the insulator 120 and some part of the light is reflected by the insulator 120. The refractive index of the walls of the fiber optic-based sensing element 130 and the geometry of the FBGs 140 changes over time as the insulator 120 absorbs moisture, and the change in the refractive index and/or geometry of the sensing element 130 causes the amplitude and/or frequency of light reflected by the FBGs 140 to change. This change can be evaluated in comparison to reflected light from the same or similar FBGs proximate to an insulator that does not include any moisture content or has a known baseline moisture content. Optical signals based on the reflected light are coupled back into the core of the fiber optic-based sensing element 130 and transmitted in return to the interrogator. The reflected optical signals are conveyed to the interrogator and the spectral response of the optical signals, including a frequency response and/or amplitude response of the optical signals, is then processed at the processing circuitry to determine the moisture content of the insulator 120. In sum, light injected into the fiber optic-based sensing element 130 is reflected back from the FBGs with a specific spectral response (including amplitude response and/or frequency response) that allows moisture absorption to be calculated by measuring changes in the spectral response, e.g., from a baseline or reference (e.g., “no moisture”) spectral response to a current spectral response.

In some embodiments, the processing circuitry may store various baseline or reference values of the spectral response of a fiber optic-based sensing element in order to assess changes in the spectral response and thereby accurately determine the moisture content of the insulator at the location where the moisture is sensed, based on a known relationship between moisture content in the insulator and changes in the spectral response. These reference values are compared with the spectral response of the fiber optic-based sensing element 130 positioned at a location sufficiently adjacent to or in immediate proximity of the insulator where moisture content needs to be sensed or measured.

In at least one embodiment, the processing circuitry uses a memory coupled to the processing circuitry to store a baseline spectral response of a fiber optic-based sensing element before it is placed inside a transformer. In another embodiment, the processing circuitry stores a spectral response of a fiber optic-based sensing element when it is placed adjacent to a dry insulator containing no moisture as a reference value.

In yet another embodiment, the processing circuitry stores a spectral response of a ‘first’ fiber optic-based sensing element that is placed adjacent to a ‘second’ fiber optic-based sensing element which is placed at a location of the insulator where moisture content needs to be measured. The first fiber optic-based sensing element may include a moisture-isolated sensing portion that is used as a reference and is placed in close proximity to the second fiber optic-based sensing element such that conditions and factors besides the moisture content in the insulator are substantially identical. The interrogator may transmit light to both the first fiber optic-based sensing element and the second fiber optic-based sensing element so that the interrogator may receive the different optical signals having different spectral responses from the two fiber optic-based sensing elements in order to compare, calculate, and determine the moisture content of the insulator at the sensed location. Further example methods of determining the moisture content of the insulator 120 use a correlation of the amount of moisture to the received optical signals.

The benefit of having a ‘reference’ or ‘isolated’ fiber optic-based sensing element is that an insulator such as paper ages over time. The paper will change as it gets older and also from the stress and ingress/outgress of moisture. Further, aging of the paper can be related directly to the heating and cooling cycles of the transformer. Accordingly, a hermetically sealed piece of insulator paper around the optical fiber can serve as a reference as the paper in the sealed portion is never in contact with the oil, but it is subject to the same heating and cooling cycles of the transformer.

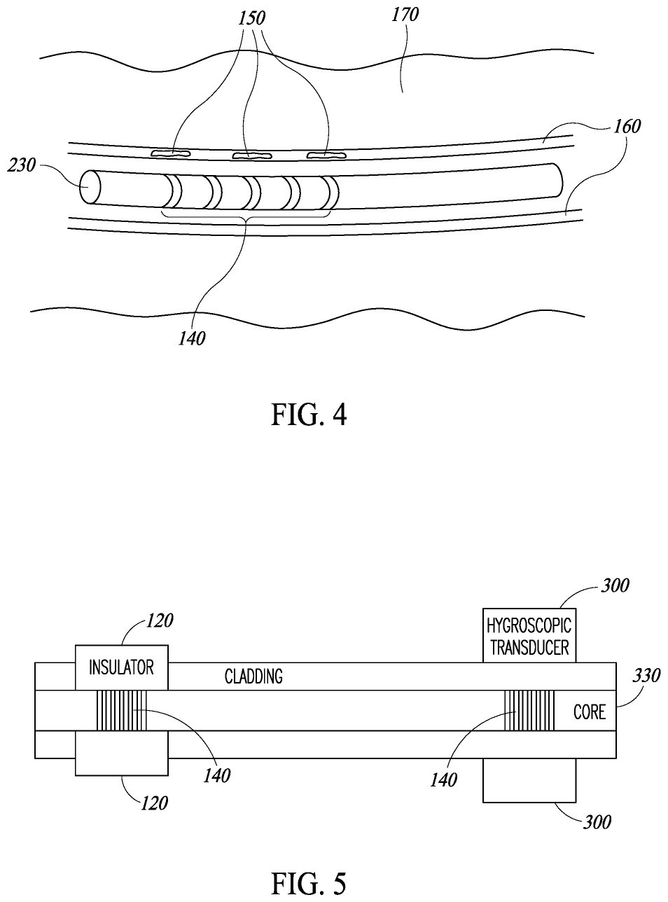

FIG. 4 is a view of using a fiber optic-based sensing element having a hygroscopic material around the fiber optic-based sensing element inside an oil-filled transformer.

FIG. 4 illustrates a different embodiment from FIG. 3 in that the fiber optic-based sensing element in FIG. 4 has a hygroscopic material 160 around the fiber optic-based sensing element whereas the fiber optic-based sensing element in FIG. 3 does not have the hygroscopic material. However, the fiber optic-based sensing element 130 in FIG. 3 and the fiber optic-based sensing element 230 in FIG. 4 are similar in the sense that FBGs 140 are provided along a length of the fiber optic-based sensing element.

According to one embodiment, the hygroscopic material 160 is provided along a length of the fiber optic-based sensing element 230. When the fiber optic-based sensing element 230 is placed inside the oil-filled transformer, the moisture 150 inside the oil 170 is absorbed into the hygroscopic material 160. This causes the spectral response of optical signals reflected by the FBGs 140 of the fiber optic-based sensing element 230 to change due to the absorption of moisture by the hygroscopic material 160. For example, the walls of the fiber optic-based sensing element 230 including hygroscopic materials 160 may absorb moisture over time, which causes changes in the spectral response of the optical signals reflected at the location where moisture is absorbed. That is, by choosing hygroscopic materials with stable optical properties for the fiber, it is possible to detect the moisture level in the surrounding oil as moisture permeates into the walls of the fiber optic-based sensing element 230 thus changing its refractive index and/or the geometry of the grating element (e.g., FBGs). This change in the optical and geometrical properties of the grating can be measured by measuring changes in reflected light amplitude and frequency by the grating element.

When the fiber optic-based sensing element 230 is first placed inside the oil-filled transformer, the interrogator may inject light into the fiber optic-based sensing element 230 and receive reflected optical signals in return. A baseline measurement of the spectral response of the reflected optical signals may be stored as a reference. As time passes and moisture 150 inside the oil 170 is absorbed into the hygroscopic material 160, the interrogator may inject light into the fiber optic-based sensing element 230 at a different point in time and receive optical signals with a different spectral response. The amount of moisture content in the insulator at that sensed location can be calculated by measuring the delta deviation of the spectral response from the baseline spectral response.

More specifically, when an interrogator (see FIG. 10) transmits light to the fiber optic-based sensing element 230, the transmitted light is leaked to the vicinity and to its surroundings due to the FBGs. A part of the light is absorbed by the hygroscopic material 160 and a part of the light is reflected by the hygroscopic material 160. The reflection of light by the hygroscopic material 160 changes over time as it absorbs moisture and the refractive index of the hygroscopic material 160 changes. The reflected light is coupled back to the core of the fiber optic-based sensing element 130 and is transmitted to the interrogator as optical signals having a measurable spectral response (e.g., frequency response and/or amplitude response).

Another method of determining the moisture content of the oil using the hygroscopic material 160 is to use an isolated fiber optic-based sensing element with hygroscopic materials. In at least one embodiment, an isolated fiber optic-based sensing element (or also referred to as “reference fiber optic-based sensing element”) indicates a fiber optic-based sensing element sufficiently adjacent to the fiber optic-based sensing element 230 to function as a reference for comparison but is sealed from moisture of the surrounding environment. Alternatively, another fiber optic-based sensing element with hygroscopic materials that is isolated may be placed in close proximity to the fiber optic-based sensing element 230. To be specific, at least one of the gratings of the isolated fiber optic-based sensing element is isolated from the sensing environment (e.g., transformer oil) and thereby preserving it as a reference which is used to detect any changes to the gratings which are exposed to the transformer oil.

The difference in spectral response (e.g., frequency response and amplitude response) for both fiber optic-based sensing elements is compared and calculated by the processing circuitry to determine the moisture content of the oil at a specific sensed location based on the moisture absorbed in the hygroscopic material 160. In other words, the difference in spectral response changes between a spectral response of reference fiber optic-based sensing element having the hygroscopic material and a spectral response of the fiber optic-base sensing element 230 having the hygroscopic material can be used to determine the moisture content in the oil at a specific sensed location.

The spectral response of a reference fiber optic-based sensing element may be obtained in many ways. The processing circuitry may store in the memory and retrieve from the memory various reference values of the spectral response of a fiber optic-based sensing element. The spectral response of a fiber optic-based sensing element having a hygroscopic material can be calculated and stored as a reference value before the fiber optic-based sensing element is placed inside a transformer. In another embodiment, the reference fiber optic-based sensing element having a sealed portion of the hygroscopic material can be placed adjacent to a fiber optic-based sensing element having a hygroscopic material that is used for sensing moisture content at a specific location within the transformer (e.g., any location within the oil or the winding). The reference fiber optic-based sensing element is placed in close proximity to the other fiber optic-based sensing element used to sense moisture content such that other conditions and factors are not substantially different. The interrogator may transmit light to both fiber optic-based sensing elements including the reference fiber optic-based sensing element so that the interrogator may receive the different spectral responses of the two fiber optic-based sensing elements in order to compare, calculate, and determine the moisture content of the oil at the sensed location based on the moisture absorbed in the hygroscopic material.

Although the hygroscopic material is shown as completely surrounding the fiber optic-based sensing element along the length of the fiber optic-based sensing element, the embodiment of the present disclosure is not limited to the embodiment shown in FIG. 4.

In another embodiment, the hygroscopic material may be used in an area only where the FBGs are located. That is, the hygroscopic material may be disposed to surround and overlap the area where the FBGs are located in the fiber optic-based sensing element.

If there are several FBGs at sensing locations along the length of the fiber optic-based sensing element that are spaced apart from each other, the same or different hygroscopic materials may be used to surround the FBGs at each sensing location. Accordingly, the hygroscopic materials may not be located in an area where the FBGs are not disposed.

According to some embodiments, the cladding of the fiber optic-based sensing element may be entirely or at least partially made of hygroscopic materials. On the other hand, according to other embodiments, the hygroscopic material may be coated on the cladding as a separate layer. However, in both cases, the spectral response that changes based on the hygroscopic material absorbing moisture is detected by the interrogator in reflected optical signals and the processing circuitry coupled to the interrogator may determine the moisture content of the oil based on the detected change in spectral response.

Examples of hygroscopic materials include, but are not limited to, Poly(methyl methacrylate) PMMA, Polysulfonate, or the like.

FIG. 5 is a cross-sectional view of a fiber optic-based sensing element 330 in a length direction of the optical fiber, including hygroscopic transducers coupled to the gratings inscribed in the core of the fiber optic-based sensing element according to some embodiments of the present disclosure.

As shown, FIG. 5 describes an additional method and system of integrating hygroscopic transducers 300 with the gratings (e.g., Fiber Bragg gratings 140) to convert structural changes induced in such materials due to moisture absorption or adsorption into stresses which, when applied to the gratings, change the spectral response induced by the gratings in reflected optical signals and thus lead to a measurable moisture content using the fiber optic-based sensing element 330.

Embodiments of FIG. 5 utilize the concept of using intrinsic hygroscopic fiber properties for moisture detection in addition to combining multiple transducers with the grating.

The moisture content of an insulator 120 may be directly measured when the insulator 120 is wrapped around gratings (e.g., FBGs 140) in a fiber optic-based sensing element. When an insulator 120 is wrapped around the FBGs 140 as shown in FIG. 5, the optical interface between the FBGs 140 and the insulator 120 is critical. An optical coupling and a mechanical coupling between the interface of the FBGs 140 and the insulator 120 allows for measurement of the moisture content in the insulator 120 by way of changes in the spectral response of optical signals reflected by the FBGs 140.

It may also be beneficial to use a hygroscopic transducer 300, as shown in FIG. 5, which has a strong bond between the fiber optic-based sensing element 330 and the hygroscopic transducer 300. The hygroscopic transducer 300 comprise of hygroscopic materials that expands and contracts in response to moisture absorption and/or adsorption. In one embodiment, the hygroscopic transducer 300 is a wrapping of the hygroscopic material 160 at a location as shown in FIG. 5, where the hygroscopic transducer 300 overlaps with the location of the grating sensor 140.

One example of bonding the hygroscopic transducer 300 to the fiber optic-based sensing element 330 includes, but are not limited to, 3D printing, gluing, fused interface, PVD, CVD, or the like.

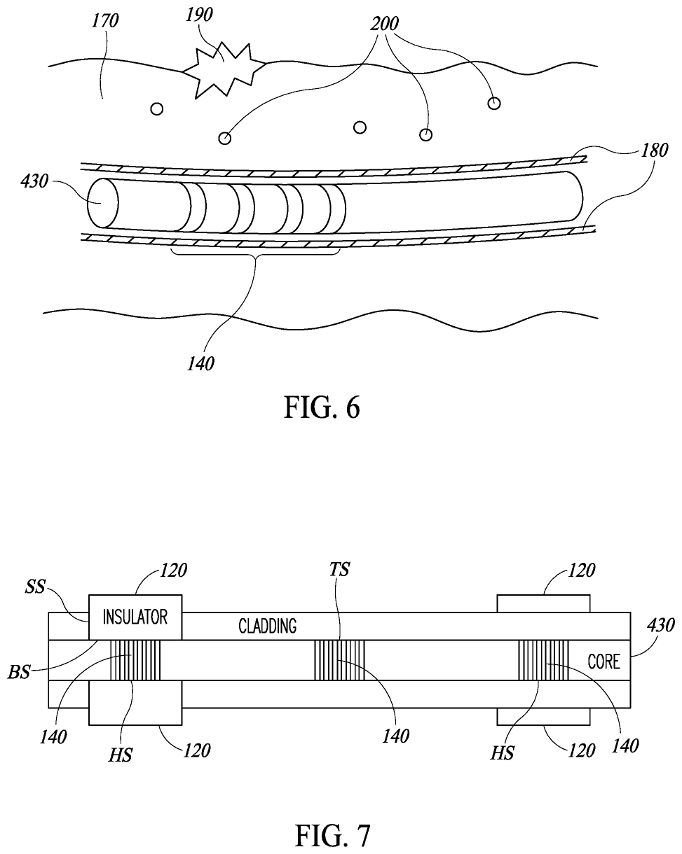

FIG. 6 is a view of using a fiber optic-based sensing element having a hydrogen sensitive material around the fiber optic-based sensing element inside an oil-filled transformer.

FIG. 6 is a different embodiment from FIG. 4 in that the fiber optic-based sensing element in FIG. 6 has a hydrogen sensitive material 180 around the fiber optic-based sensing element whereas the fiber optic-based sensing element in FIG. 4 has a hygroscopic material 160 instead. However, the fiber optic-based sensing element 230 in FIG. 4 and the fiber optic-based sensing element 430 in FIG. 6 are similar in the sense that FBGs are provided along a length of the fiber optic-based sensing element.

As described in connection to the different embodiments in FIGS. 3 to 5, a fiber optic-based sensing element having FBGs can be used to measure various environmental parameters, such as moisture. The fiber optic-based sensing elements can be extended to measure other parameters or additional parameters such as pressure, vibrations, liquid levels, and hydrogen in electrical transformers.

Internal arcing in an oil-filled electrical transformer can instantly vaporize surrounding oil, generating gas pressures that can cause rupture in the tank and potentially spread flaming oil over a large area. Oil preservation system malfunctions can increase the operating pressure above the threshold. Therefore, it is beneficial to monitor pressure inside a transformer to avoid catastrophic failures and outages.

In some embodiments, this pressure when applied to FBG gratings, changes the spectral response of light reflected by the FBG gratings, and thus lead to a measurable pressure parameter based on the reflected light in the fiber optic-based sensing element.

Another cause of transformer failure is dielectric breakdown of the transformer's insulation system. These failures are often preceded by partial discharge activity (e.g., electrical sparks). Partial discharges in oil produces hydrogen dissolved in the oil.

According to various embodiments as shown in FIG. 6, a coating with a hydrogen sensitive material 180 can be applied to the outer surface of the optical fiber where the FBGs are located. In some cases, hydrogen sensitive material can expand or contract in response to the presence of hydrogen. When the hydrogen sensitive material expands and creates strain, this changes the geometry of the FBGs, which changes the spectral response of light reflected by the FBGs, and lead to a measurable parameter that is processed to determine hydrogen concentration in transformer oil.

According to at least one embodiment, the hydrogen sensitive material 180 is provided along a length of the fiber optic-based sensing element 430. Although the hydrogen sensitive material 180 is shown as completely surrounding the fiber optic-based sensing element 430 along the length of the fiber optic-based sensing element 430, the present disclosure is not limited to the embodiment shown in FIG. 6.

For instance, in another embodiment, the hydrogen sensitive material 180 may surround only an area where the FBGs are located. That is, the hydrogen sensitive material 180 may be disposed to surround and overlap the area where the FBGs are located in the fiber optic-based sensing element 430.

If there are several FBGs along the length of the fiber optic-based sensing element 430 that are spaced apart from each other, the hydrogen sensitive material 180 may surround each of the FBGs at their respective locations. Accordingly, the hydrogen sensitive material 180 may not be located in an area where the FBGs are not disposed.

According to some embodiments, the cladding of the fiber optic-based sensing element may be entirely or at least partially made of a hydrogen sensitive material 180. On the other hand, according to other embodiments, the hydrogen sensitive material 180 may be coated on the cladding as a separate layer. However, in both cases, the hydrogen sensitive material 180 absorbing hydrogen 200 affects the geometry of the FBGs which causes changes in the spectral response of optical signals reflected by the FBGs. This change in spectral response is sensed by the interrogator and communicated to the processing circuitry coupled to the interrogator to determine the hydrogen content of the oil in the transformer.

FIG. 7 is a cross-sectional view of a fiber optic-based sensing element having an insulator wrapped around and affixed to gratings (e.g., FBGs) according to various embodiments of the present disclosure.

As shown, a transformer insulation paper (one example of an insulator 120) is wrapped around and affixed to an FBG inscribed into a silica fiber. As the optical properties of the insulation paper change due to moisture adsorption from the surrounding oil, the spectral response (amplitude and/or frequency) of light reflected and transmitted by the fiber optic-based sensing element 430 changes, producing measurable parameters which in turn indicate the amount of moisture in the transformer oil.

The fiber optic-based sensing element 430 can implement different type of sensors based on the FBGs 140. Further, different types of sensor may be implemented along different portions of the same fiber optic-based sensing element. In the example fiber optic-based sensing element 430 shown in FIG. 7, humidity sensors HS are implemented using FBGs 140 that are inscribed into the fiber optic-based sensing element 430 at two locations as shown, where the insulator is wrapped around and affixed to the FBGs. The fiber optic-based sensing element 430 also includes a temperature sensor TS between adjacent humidity sensors HS. Here, in this example, the temperature sensor TS is not surrounded by the insulator 120. In at least one embodiment, the interrogator can determine which reflected optical signals are received from which of the sensors (the two humidity sensors HS or the temperature sensor TS) by evaluating time of transmission of the injected interrogating light to time of receipt of the reflected optical signals. Light reflected by a sensor that is farther along the length of the fiber optic-based sensing element 430 will take a longer time than light reflected by a sensor that is closer to the interrogator.

In at least one embodiment, the insulator 120 can be positioned on and affixed to the cladding of the fiber optic-based sensing element 430. In another embodiment, the insulator 120 can be positioned on and affixed to the core of the fiber optic-based sensing element 430. That is, a bottom surface BS of the insulator 120 may directly contact the core (and the FBGs in that location) and a side surface SS of the insulator 120 may directly contact the cladding of the fiber optic-based sensing element 430. FIG. 7 illustrates both embodiments.

The method of measuring the moisture content is determined in a similar manner to the method described in connection with FIG. 3 or 4. That is, the fiber optic-based sensing element 430 receives interrogating light from an interrogator. At least a portion of the light injected into the fiber optic-based sensing element 430 from the interrogator is reflected by the FBGs 140. The amplitude response and the frequency response of the reflected light, as they change according to moisture absorption, can be detected and calculated by the interrogator (or by the processing circuitry connected to the interrogator). By comparing the spectral response of the reflected optical signals with a reference or baseline spectral response, and measuring the delta deviation of the spectral response of the reflected optical signals, the moisture content can be determined.

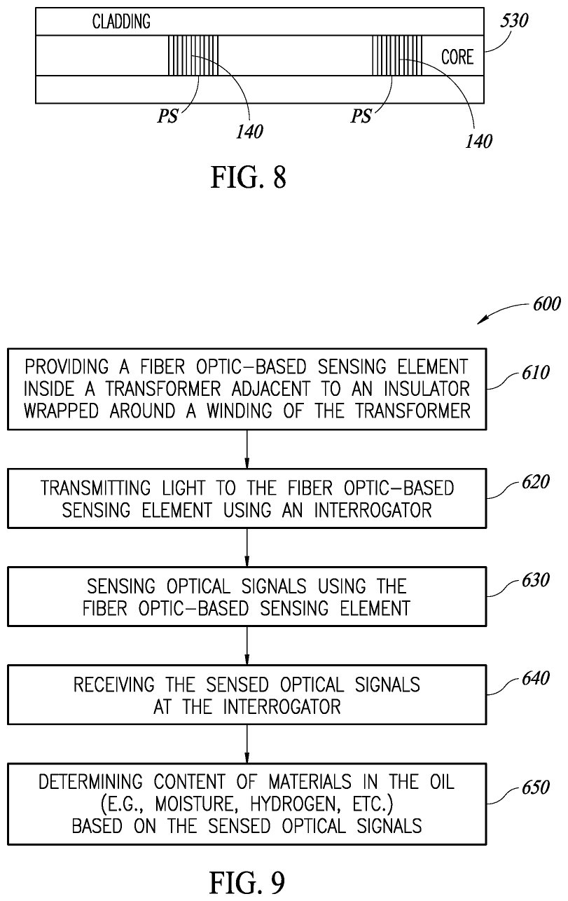

As shown, FBGs can be implemented in many ways to sense different parameters including pressure. FIG. 8 illustrates a fiber optic-based sensing element using gratings for a pressure sensor according to some embodiments of the present disclosure.

In a new transformer, the windings are assumed to be under a clamping force that is equal or greater to the forces developed during a through-fault. However, as aging takes place, the insulation 120 of the transformer 100 loses some of its mechanical properties and shrinkage takes place. This reduces gradually the windings' initial clamping force resulting in mechanical looseness of the windings 110 making the windings 110 prone to fail or subject to deformations under through-fault conditions. Thus, as long as the transformer's clamping system (not shown) maintains the clamping pressure, the windings may remain tight during a through fault event and should therefore not sustain any damage, due to movement of the conductors. The issue becomes problematic to transformer operators in that when a transformer ages, the winding relaxes and becomes loose.

Generally, transformers have a rigid clamping system to compress the winding to a specified pressure. Any change in the thickness of the materials in the winding 110 and associated insulation may change the pressure on the winding 110. The thickness of the conductor material will not change except for the thermal expansion and contraction due to changes in temperature. The cellulose insulation material, being organic, will change in thickness and elasticity over time resulting from the effect of moisture, temperature, and aging. Therefore, it is beneficial to measure the clamping pressure to detect in a timely manner the looseness of windings 110 to avoid a failure of a transformer 100 during a through fault.

Additionally, it is note that twelve to fifteen percent of transformer failures are caused by winding deformations. These geometric variations lead to an increase of winding vibration and, consequently, to an increase of the solid insulation mechanical fatigue. The insulation can be degraded and short circuits between turns may appear. These winding deformations can also change the distance between conductors, changing the windings series and shunt capacitances. In these cases, the voltage distribution in case of lightning or switching over voltages are changed and is different from what the transformer was designed to withstand, and therefore increases the risk of failure. Also, measuring the vibration of the core provides information on whether the vibration of the coils is caused by the core, which can be caused by the core being loose. Therefore, there are several technical benefits to using a fiber optic-based sensing element as described herein to directly measure vibrations to detect winding deformations and/or a loose core.

In some embodiments, FBG sensors 140 can be disposed directly in the windings and the core inside an oil-filled power transformer tank to measure the vibration of the core and the windings 110 by measuring strain force on the FBGs. FIG. 8 shows a pressure sensor PS implemented based on FBGs 140 in a fiber optic-based sensing element 530 which can measure clamping pressure of the winding. For instance, clamping pressure can be measured by affixing the FBGs 140 to the section of the winding near the clamp. Vibrations causing changes in the geometry of the FBGs 140 can be detected by changes of spectral response in light reflected by the FBGs, as described herein, and these spectral changes can be correlated with changes in pressure on the FBGs 140, thus allowing the measurement system to measure vibrations over time. Another example method for measuring clamping pressure is to measure strain at the clamp by affixing the FBGs 140 of the fiber optic-based sensing element 530 to the body of the clamp.

The application of the fiber optic-based sensing element 530 having pressure sensors PS can be used in different settings to detect different signals and parameters. For instance, failures in load tap changers are frequently caused by faults that are mechanical in nature. These include failures of springs, bearings, shafts, and drive mechanisms. In some embodiments, measuring the strain forces using FBGs 140 which change their spectral response and thus lead to a measurable changes in light reflected by the FBGs to an interrogator can be used to monitor vibrations in load tap changers. Some embodiments of the present disclosure can also measure clamping pressure of an on-line transformer. Different types of fiber materials and inscriptions can be used such as FBG, LPG and Fabry-Perot to achieve the various objectives described herein, according to different applications of the fiber optic-based sensing element. For example, the type of optical fiber used for measuring strain forces may be different than the type of optical fiber used for measuring temperature and/or humidity. The optical fiber used for strain, for example, may be a silica or sapphire fiber coupled with a strain gauge.

FIG. 9 is a flow chart of a method according to various embodiments of the present disclosure.

The method 600 includes providing, at 610, a fiber optic-based sensing element inside a transformer at a first location adjacent to an insulator wrapped around a winding of the transformer. The fiber optic-based sensing element includes at least one set of gratings (e.g., Fiber Bragg gratings) along a length of the fiber optic-based sensing element. At 620, the method includes transmitting light through the fiber optic-based sensing element using an interrogator. At 630, the method includes sensing optical signals reflected from the set of gratings. At 640, the reflected optical signals are sensed by the interrogator. The method 600 concludes at 650 by determining a sensed parameter (e.g., amount of moisture, hydrogen, or the like) based on the sensed optical signals, including for example changes in spectral response of the sensed optical signals.

As described throughout the present disclosure, embodiments of the present disclosure provide methods for directly measuring an environmental parameter, such as moisture content, in paper insulation or the insulating oil in the transformer. This method allows for measurement of environmental parameters (e.g., moisture content) at multiple specific locations (e.g., point by point assessment) in the transformer.

FIG. 10 illustrates a system according to various embodiments of the present disclosure.

The system 700 includes a transformer 100, an interrogator 210, and processing circuitry 750. Other elements not shown in FIG. 10 may also be included in the system 700 such as a memory (not shown) coupled to the processing circuitry 750.

A fiber optic-based sensing element 130 is positioned adjacent to a location in the transformer 100 where a desired parameter is to be measured. An interrogator 210 coupled to an end of the fiber optic-based sensing element 130 sends interrogating light through the fiber optic-based sensing element 130 to a grating sensor, such as an FBG sensor 140, positioned at the location in the transformer 100 to be measured. The interrogator 210 receives reflected light from the grating sensor. Measurement data based on quantified characteristics of the reflected light (e.g., spectral response) as determined by the interrogator 210 may be processed at the processing circuitry 750 to provide information on physical parameters of the transformer (or an environment within the transformer) at the sensed location, for generating maintenance reports and the like. In some embodiments, measurement data may refer to raw data measured from light reflected by each grating sensor, which could be changed in the frequency content or amplitude of the reflected light from each grating sensor. The measurement data is then correlated to, for example, the stress exerted by pressure sensitive material wrapped around the grating sensor or it could be changed in the frequency content or amplitude of the reflected light as a result of the physical changes in paper insulation 120 wrapped around or directly adjacent to the grating sensor, as the paper insulation 120 absorbs moisture for example.

The processing circuitry 750 may be electrically connected (e.g., by a USB cable) to the interrogator 210 and receives measurement data (e.g., measured light characteristics) from the interrogator 210. The processing circuitry 750 determines the physical parameters based on the measured light characteristics. These parameters may be used to calculate valuable maintenance data such as PPM (parts per million) of moisture content in the transformer oil or moisture content in the paper insulation 120 of windings 110 in the transformer 100.

In various embodiments, measurement data from an interrogator 210 may be communicated wired or wirelessly to the processing circuitry 750.

One or more embodiments of the direct measurement method using fiber optic grating sensors according to the present disclosure reduces the need for additional cables to measure each parameter (e.g., as used in current fiber optic sensors with semiconductor terminations). The direct measurement method using fiber optic grating sensors also provides better immunity to electromagnetic interference EMI (e.g. electrical sensing elements). It also provides higher accuracy (using spectral measurements) and reduces overall cost of installation.

Further, the ability to measure multiple parameters simultaneously presents the possibility to diagnose transformer faults more holistically based on stochastic models. Embodiments of the present disclosure can advantageously reduce false alarms and human errors in diagnosing asset health, and ultimately automate predictive maintenance and proactively repair and replace assets before they fail.

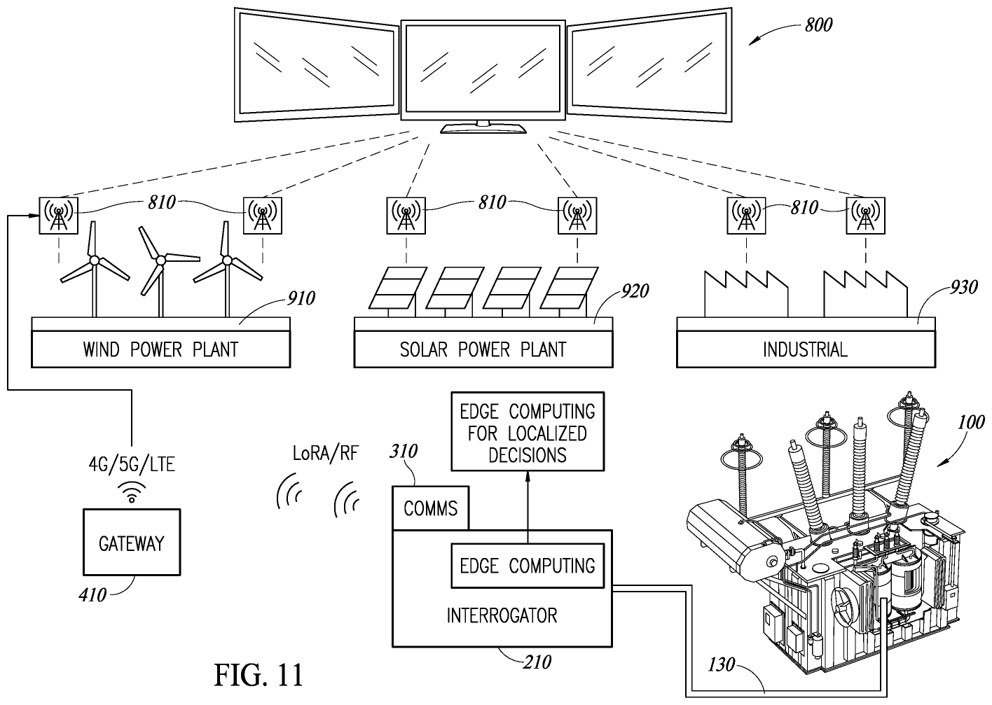

FIG. 11 illustrates an example of a monitoring system according to various embodiments of the present disclosure.

A fiber optic-based sensing element 130 in a transformer 100 is coupled to an interrogator 210. The interrogator 210 receives reflected optical signals from the fiber optic-based sensing element 130 and based thereon, the interrogator 210 determines a physical parameter of the transformer 100 or an environment within the transformer 100. Such physical parameter may include, for example, a level of moisture or hydrogen in the transformer oil or insulating paper. The interrogator 210 is operatively coupled to a communication unit 310 so that parameter information (e.g., the moisture content information or the hydrogen content information) can be wirelessly transmitted. A monitoring system 800 also includes a gateway 410 communicatively coupled to a communication tower 810 so that the information from various plants (e.g., wind power plant 910, solar power plant 920, industrial plants 930, or the like) using transformers, such as the transformer 100, can be transmitted real-time to the monitoring system 800.

In some embodiments, communication technologies such as LTE/4G, 5G, and Ethernet may be used to provide an IOT (Internet of Things) gateway solution for remote monitoring as shown in FIG. 11. In some embodiments, the interrogator 210 may include an edge computing device for localized decisions.

One or more embodiments of the present disclosure relate to online measurement of moisture in paper insulation, temperature, and clamping pressure, or a combination of these parameters, in a transformer using a fiber optic-based sensing element as described herein. For instance, a polymer optical fiber with a grating sensor as described herein may be used to assess the health of electrical transformers and other assets in high voltage environment. The embodiments described herein can further be utilized to measure rapid pressure rise in the transformer tank, liquid level, vibration in windings, hydrogen levels, and etc.

Embodiments of the present disclosure may facilitate providing significant improvements in asset monitoring and maintenance, including computational costs and reduced delay.

Some embodiments may take the form of or comprise computer program products. For example, according to an embodiment of the present disclosure, there is provided a computer readable medium comprising a computer program adapted to perform one or more of the methods or functions described above. The medium may be a physical storage medium, such as for example a Read Only Memory (ROM) chip, or a disk such as a Digital Versatile Disk (DVD-ROM), Compact Disk (CD-ROM), a hard disk, a memory, a network, or a portable media article to be read by an appropriate drive or via an appropriate connection.

Furthermore, in some embodiments, some or all of the methods and/or functionality may be implemented or provided in other manners, such as at least partially in firmware and/or hardware, including, but not limited to, one or more application-specific integrated circuits (ASICs), digital signal processors, discrete circuitry, logic gates, standard integrated circuits, controllers (e.g., by executing appropriate instructions, and including microcontrollers and/or embedded controllers), field-programmable gate arrays (FPGAs), complex programmable logic devices (CPLDs), etc., and various combinations thereof.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
providing a fiber optic-based sensing element inside a transformer at a first location adjacent to an insulator wrapped around a winding of the transformer;
transmitting light through the fiber optic-based sensing element;
sensing optical signals based on light reflected by a grating sensor defined along a length of the fiber optic-based sensing element at the first location; and
determining a moisture parameter of the insulator wrapped around the winding at the first location based on the sensed optical signals.

2. The method of claim 1, wherein the insulator includes cellulose paper.

3. The method of claim 1, wherein providing the fiber optic-based sensing element inside the transformer at the first location adjacent to the insulator includes:
positioning the grating sensor either in immediate proximity to or in direct contact with the insulator wrapped around the winding of the transformer.

4. The method of claim 1, wherein moisture absorbed in the insulator changes a physical parameter of the insulator at the first location, and
wherein sensing optical signals based on light reflected by the grating sensor includes:
determining a spectral response of the optical signals based on the light reflected by the grating sensor,
wherein a change in the spectral response of the optical signals is indicative of the moisture parameter of the insulator at the location.

5. The method of claim 1, wherein determining the moisture parameter of the insulator wrapped around the winding at the first location includes:
calculating a change in spectral response of the sensed optical signals from a reference spectral response to a current spectral response.

6. The method of claim 5, wherein calculating the change in spectral response of the sensed optical signals includes:
determining the reference spectral response at a first point in time;
determining the current spectral response at a second point in time after the first point in time; and
calculating a difference in the spectral response from the reference spectral response to the current spectral response.

7. The method of claim 6, wherein determining the moisture parameter of the insulator wrapped around the winding at the first location further includes:
determining the moisture parameter of the insulator based on a known relationship between moisture content in the insulator and changes in the spectral response of the optical signals.

8. The method of claim 5, further comprising obtaining the reference spectral response from one or more fiber optic gratings that are positioned inside the transformer adjacent to the insulator but isolated from moisture in the transformer.

9. The method of claim 8, further comprising:
providing a second fiber optic-based sensing element inside the transformer at a second location adjacent to the insulator wrapped around the winding of the transformer;
sensing optical signals based on light reflected by the grating sensor defined at the first location and by a second grating sensor defined along a length of the second fiber optic-based sensing element at the second location; and
determining a moisture parameter of the insulator wrapped around the winding at the first location and at the second location based on the sensed optical signals including light reflected by the grating sensor at the first location and the second grating sensor at the second location.

10. A method, comprising:
providing a fiber optic-based sensing element inside an insulating oil of an oil-filled transformer, the fiber optic-based sensing element having a light-transmitting core and a hygroscopic material at least partially surrounding the light-transmitting core;
transmitting light through the fiber optic-based sensing element;
sensing optical signals that are reflected back from a grating sensor defined in the light-transmitting core at a location along a length of the fiber optic-based sensing element;
determining a moisture parameter of the insulating oil of the oil-filled transformer based on the sensed optical signals.

11. The method of claim 10, wherein the hygroscopic material, in operation, absorbs moisture in the oil, and an absorption of moisture by the hygroscopic material changes a refractive index of the fiber optic-based sensing element at the location where the hygroscopic material at least partially surrounds the light-transmitting core of the fiber optic-based sensing element.

12. The method of claim 11, wherein sensing optical signals that are reflected back from the grating sensor includes:
sensing a frequency response and an amplitude response of the optical signals,
wherein the frequency response and the amplitude response of the optical signals are indicative of the moisture parameter at the location where the hygroscopic material at least partially surrounds the light-transmitting core of the fiber optic-based sensing element.

13. The method of claim 12, wherein determining the moisture parameter of the insulating oil of the oil-filled transformer includes:
determining the moisture parameter of the insulating oil based on a change in the frequency response and/or the amplitude response from a reference frequency response and/or a reference the amplitude response.

14. The method of claim 12, wherein determining the moisture parameter of the insulating oil of the oil-filled transformer further includes:
comparing the frequency response and the amplitude response of the fiber optic-based sensing element having the hygroscopic material with a reference frequency response and a reference amplitude response of a second fiber optic-based sensing element not having a hygroscopic material,
wherein the second fiber optic-based sensing element is disposed adjacent to the fiber optic-based sensing element having the hygroscopic material.

15. A system, comprising:
a first fiber optic-based sensing element at a first location inside an oil-filled transformer, the first fiber optic-based sensing element including at least one set of Fiber Bragg gratings defined along a length of the first fiber optic-based sensing element at the first location;
a second fiber optic-based sensing element at a second location inside the oil-filled transformer, the second fiber optic-based sensing element including at least one set of Fiber Bragg gratings defined along a length of the second fiber optic-based sensing element at the second location;
an interrogator operatively coupled to the first and second fiber optic-based sensing elements, the interrogator configured to:
transmit interrogating light to the first and second fiber optic-based sensing elements; and
receive optical signals based on light reflected back from the first and second fiber optic-based sensing elements;
a processing circuitry coupled to the interrogator, the processing circuitry configured to:
calculate a change in spectral response of the optical signals from a first point in time to a second point in time after the first point in time; and
determine a moisture parameter at the first location or at the second location based on the change in the spectral response of the optical signals.

16. The system of claim 15, wherein the first fiber optic-based sensing element includes an insulator wrapped around a circumference of the first fiber optic-based sensing element,
wherein the insulator overlaps the set of Fiber Bragg gratings of the first fiber optic-based sensing element,
wherein, in operation, the insulator absorbs moisture in the transformer, and
wherein the change in the spectral response of the optical signals from the first point in time to the second point in time is indicative of the moisture absorbed by the insulator during a period between the first point in time and the second point in time.

17. The system of claim 15, wherein the first fiber optic-based sensing element includes a layer of hygroscopic material coated around a circumference of the first fiber optic-based sensing element,
wherein the layer of the hygroscopic material overlaps the set of Fiber Bragg gratings of the first fiber optic-based sensing element,
wherein, in operation, the layer of the hygroscopic material absorbs moisture in the transformer which causes a change in the set of Fiber Bragg gratings of the first fiber optic-based sensing element, and
wherein the change in the spectral response of the optical signals from the first point in time to the second point in time is indicative of moisture absorbed by the hygroscopic material during a period between the first point in time and the second point in time.

18. The system of claim 15, further comprising a hygroscopic transducer,
wherein the hygroscopic transducer is coupled to the first fiber optic-based sensing element at an outer surface of the first fiber optic-based sensing element,
wherein the hygroscopic transducer overlaps the set of Fiber Bragg gratings of the first fiber optic-based sensing element,
wherein, in operation, a physical parameter of the hygroscopic transducer changes relative to changes in moisture in the transformer and a change in the physical parameter of the hygroscopic transducer causes a change in the set of Fiber Bragg gratings of the first fiber optic-based sensing element, and
wherein the change in spectral response of the optical signals from the first point in time to the second point in time is indicative of the moisture in the transformer at the first location during a period between the first point in time and the second point in time.

19. The system of claim 15, wherein the first fiber optic-based sensing element includes a layer of hydrogen sensitive material coated around the first fiber optic-based sensing element,
wherein the layer of the hydrogen sensitive material overlaps the set of Fiber Bragg gratings of the first fiber optic-based sensing element,
wherein, in operation, the layer of the hydrogen sensitive material absorbs hydrogen in the transformer which causes a change in the set of Fiber Bragg gratings of the first fiber optic-based sensing element, and
wherein the change in the spectral response of the optical signals from the first point in time to the second point in time is indicative of hydrogen absorbed by the hydrogen sensitive material at the first location during a period between the first point in time and the second point in time.

20. The system of claim 15, wherein the first fiber optic-based sensing element including the at least one set of Fiber Bragg gratings is implemented as a pressure sensor configured to sense pressure in the transformer,
wherein, in operation, the pressure sensor senses a change in clamping pressure of windings of the transformer, and
wherein the change in spectral response of the optical signals from the first point in time to the second point in time is indicative of the change in clamping pressure during a period between the first point in time and the second point in time.
